(19) United States
(12) Patent Application Publication
(10) Pub. No.: US 2021/0290178 A1
(43) Pub. Date: Sep. 23, 2021

(54) BRAIN INJURY MONITORING WITH RECOVERY TRAJECTORY

(71) Applicant: Duke University, Durham, NC (US)

(72) Inventors: Bradley Kolls, Durham, NC (US); Leslie Collins, Durham, NC (US); Chandra Throckmorton, Durham, NC (US)

(21) Appl. No.: 17/260,094

(22) PCT Filed: Jul. 18, 2019
(86) PCT No.: PCT/US2019/042370

§ 371 Date: Jan. 13, 2021

Related U.S. Application Data
(60) Provisional application No. 62/701,346, filed on Jul. 20, 2018.

(51) Int. Cl. A61B 5/00; A61B 5/369; A61B 5/03; A61B 5/318; G16H 40/67; G16H 10/60; G16H 50/70; G16H 70/60; G16H 50/20; G16H 15/00; G16H 50/30; G06N 20/00

(52) U.S. Cl. CPC A61B 5/7275; G16H 40/20; A61B 5/031; A61B 5/318; A61B 5/7264; A61B 5/4064; G16H 40/67; G16H 10/60; G16H 50/70; G16H 70/60; G16H 50/20; G16H 15/00; G16H 50/30; G06N 20/00; A61B 5/369

(57) ABSTRACT

Systems and methods for continuously monitoring brain function of clinic patients. In some such implementations, a system uses continuously monitored EEG data signal and other information from multiple clinical sources to estimate the current severity of a brain injury and to predict recovery potential of a given patient with the brain injury. Healing progress can be monitored by displaying a predicted recovery trajectory indicative of the predicted recovery potential and, in some implementations, a series of estimations of the severity of the brain injury at each of a plurality of different times during the recovery process.

### RELATED APPLICATIONS

This application claims the benefit of U.S. Provisional Patent Application No. 62/701,346, filed Jul. 20, 2018, entitled “BRAIN INJURY MONITORING DEVICE,” the entire contents of which are incorporated herein by reference.

### BACKGROUND

Embodiments presented herein relate to a device for the processing of raw biometric signals to assess the severity of an injury of a patient in the context of clinical care.

Acute brain injury, for example, stroke and trauma, affects numerous individuals every day. Despite the pervasiveness of this medical condition, there are few monitoring techniques available to treating physicians that provide information on current brain function. For example, in cases of stroke, current clinical practice involves monitoring heart rhythm and blood pressure and performing a clinical neurologic exam. Current practice guidelines based on targeted thresholds for vital signs (often referred to as “vitals”) prevent custom care or the determination of the effectiveness of clinical interventions during the acute injury phase of care. For another example, therapies and interventions on neurologic injury and disease are typically measured many days (for example, 90 days) after the injury first occurred. Uncontrolled events may occur between endpoint assessment and clinical interventions.

### SUMMARY

In some embodiments, the invention provides a device for continuously monitoring brain function of clinic patients. In some such embodiments, the invention provides a system that uses the continuously monitored EEG data signal and other information from multiple clinical sources to estimate the current severity of a brain injury and to predict recovery potential of a given patient with the brain injury. Healing progress can be monitored by displaying a predicted recovery trajectory indicative of the predicted recovery potential and, in some embodiments, a series of estimations of the severity of the brain injury at each of a plurality of different times during the recovery process. In some embodiments, the predicted recovery trajectory and/or the estimation of the current severity of the brain injury are determined using machine-learning mechanisms trained to output a numerical probability for each of a plurality of different severity classes. In some such embodiments, the machine-learning mechanism includes a plurality of classifiers each trained to analyze a different data stream (e.g., text data, image data, biosignal data, etc.).

In one embodiment, the invention provides a device configured to calculate a recovery trajectory of a patient based on a severity of an injury of the patient. The device includes a communication interface, a display, and an electronic processor. The electronic processor is configured to receive clinical information regarding the patient and raw biometric data. The electronic processor analyzes the raw biometric data and the clinical information to produce a set of analytic measures and calculates a recovery trajectory based on the analytic measures. The recovery trajectory is then shown graphically on the display of the device.

In another embodiment, the invention provides a method for calculating a recovery trajectory of a patient based on a severity of an injury of the patient. Clinical information for the patient and raw biometric data is received and analyzed to produce a set of analytic measures. The analytic measures are then used to calculate a recovery trajectory that is displayed graphically on a display screen.

Other aspects of the invention will become apparent by consideration of the detailed description and accompanying drawings.

### BRIEF DESCRIPTION OF THE DRAWINGS

FIG. 1 is a block diagram of an electronic processing device in accordance with some embodiments.

FIG. 2 is a block diagram of a system including the electronic processing device of FIG. 1.

FIG. 3 is a flowchart of the method of determining a recovery trajectory of a patient based on the severity of an injury implemented by the electronic processing device of FIG. 1.

FIG. 4 is a power analysis table illustrating graphs of three frequency-based power features extracted from mouse EEG following varied periods of ischemia.

FIG. 5 is a flowchart of a machine-learning approach for determining a recovery trajectory assessment implemented by the electronic processing device of FIG. 1.

FIG. 6 is a table of one example of classifier probability outputs used in the machine-learning approach of FIG. 5.

FIG. 7 is a table of classifier weights of the probability outputs of FIG. 6 used in the machine-learning approach of FIG. 5.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Stroke and acute brain injury (ABI) management guidelines are currently centered on management of blood pressure and monitoring of vitals. In cases in which brain swelling is prominent, invasive devices like intracranial pressure monitors are recommended to allow calculation of cerebral perfusion pressures and intracranial pressures which, again, drive use of vitals-based management and guidelines. Vitals-based guidelines do not allow for personalization of care and result in untargeted management for any given brain injury patient. Accordingly, there is need for a device that allows continuous brain monitoring of a patient.

In some implementations, the systems and methods for brain monitoring (e.g., as discussed below) may be non-invasive, low cost, disposable, widely available, and easy for bedside providers to interpret and use. Systems and methods described herein provide continuous brain monitoring based on electroencephalography (EEG). Existing EEG techniques are non-invasive, and may be used to directly measure electrical activity of the brain. EEG provides continuous measurements of brain condition and may be used to measure brain function and to track changes in brain injury condition, for example, evolving ischemia, development of seizures, and prognosis in global hypoxic-ischemic injury. However, such EEG-based techniques are not automated, thus requiring highly-trained personnel to analyze the raw EEG data post hoc rather than providing real-time feedback to clinicians and providers at the bedside of the patient.

While EEG can also be represented by values of blood pressure, heart rate, and background frequencies, when this data is stored for use in complex analytical assessments, it is not possible to include the waveforms in the analysis directly. To do so would require massive computing capacity, automated process for recognizing file structures and data types, and the application of algorithms with varied accuracy at recognizing a pre-determined pattern within the waveform data. Embodiments described herein solve this complex problem by converting the waveforms into a dataset that can be queried for characteristic content without requiring direct assessment of the raw biometric data stream.

The creation and implementation of clinically relevant brain monitoring would revolutionize brain injury care by providing a direct measure of brain function to guide early management, and would allow for comparison of current brain function against baseline function and normative recovery curves, providing a foundation for more accurate prognosis and trajectory of clinical recovery. The embodiments described herein would improve clinical care practices and clinical trials in brain injury as the endpoint for therapy could be measured as an improvement in brain function and recovery trajectory in response to therapy instead of disposition at discharge or function at 90 days (which can be adversely impacted by numerous other factors unrelated to the effect of the early therapeutic intervention).

Early observations that 15-30% of adult patients with altered mental status in an intensive care unit (ICU) setting are having non-convulsive seizures has resulted in a progressive, but slow, adoption of ICU EEG for detection and management of seizures. However, more recently, ICU EEG has been found to be useful in prognosis of recovery from cardiac arrest and in the detection of evolving delayed ischemia in patients who suffer high-grade aneurysmal subarachnoid hemorrhage. While other technologies such as magnetic resonance imaging, cerebral oximetry, computed tomography, and ultrasound can provide insight into brain structure, perfusion, and in some ways function, they are costly, not continuous, spatially limited, difficult to implement in critically ill patients, and are not currently in widespread clinical practice like EEG.

Various forms of EEG analysis have been contemplated and applied in the field. Quantitative EEG analytics provide the basis for automated EEG interpretations including commercial trending software. Most quantitative EEG studies have been focused on seizure detection and epilepsy, though some have explored the use of EEG in detecting stroke, evolving ischemia in subarachnoid hemorrhage, and severity of brain injury following cardiac arrest. These successful applications of EEG demonstrate the feasibility of EEG to perform the role of brain monitor across injuries. However, current commercial products fall short in translation, as they exchange one nuanced form of pattern recognition for another with significant false positive rates in real world applications. Embodiments described herein differ in that machine-learning approaches leveraged with several analytic approaches are utilized and tailored for utility for a given injury type which allows for better characterization of the EEG signals and greater utility in detecting changes in brain function over time.

As mentioned above, current use of EEG in the ICU is limited to seizure detection and trending. The systems and methods described herein expand on the use of EEG by utilizing analytics that identify discrete brain states within the recovery process, allowing comparison of a given patient to normative recovery curves based on the changes in identified analytics over time post injury. By defining these changes in brain state over time, recovery of a patient may be tracked and predicted, deviations from good recovery may be detected, effects of therapeutic interventions may be measured, and early predictions about outcome and recovery potential of a patient may be interpreted in a way that current EEG interpretation by humans fails to do.

The systems and methods described herein allow continuous monitoring of EEG at a patient's bedside (for example, throughout the course of hospitalization), making EEG monitoring more scalable and accessible. While changes in characteristics of EEG have been previously observed to coincide with changes in brain injury severity across a variety of injuries, the current approach allows visual observation of changes in the EEG signals over time that are difficult, if not impossible, to track and quantify by even the most experienced readers providing interval offline analysis and review. Such a system allows interpretable EEG data for the non-EEG-reading bedside provider and is useful in clinical assessment and decision-making and provides a new approach to clinical trials of novel therapies and interventions in acute brain injury.

Accordingly, systems and methods described herein are directed to continuous brain monitoring of a patient. One example embodiment provides a device configured to calculate a recovery trajectory of a patient based on a severity of an injury of the patient. The device includes a communication interface, a display, and an electronic processor. The electronic processor is configured to receive, via the communication interface, clinical information regarding the patient, receive, via the biometric sensor, raw biometric data, analyze the raw biometric data and the clinical information to produce a set of analytic measures, calculate, based on the clinical information and the set of analytic measures, a recovery trajectory, and display, on the display, the recovery trajectory.

Another example embodiment provides a method for calculating a recovery trajectory of a patient. The method includes receiving, via a communication interface, clinical information regarding the patient, receiving, via a biometric sensor, raw biometric data, analyzing the raw biometric data and the clinical information to produce a set of analytic measures, calculating, based on the clinical information and the set of analytic measures, a recovery trajectory, and displaying, on a display, the recovery trajectory.

For ease of description, some or all or the example systems presented herein are illustrated with a single example of each of its component parts. Some examples may not describe or illustrate all components of the systems. Other example embodiments may include more or fewer of each of the illustrated components, may combine some components, or may include additional or alternative components. Although particular examples of biometric data are illustrated and described, it should be understood that the methods and processes described herein may be used on any time series or waveform data stream and are not limited to the examples used here.

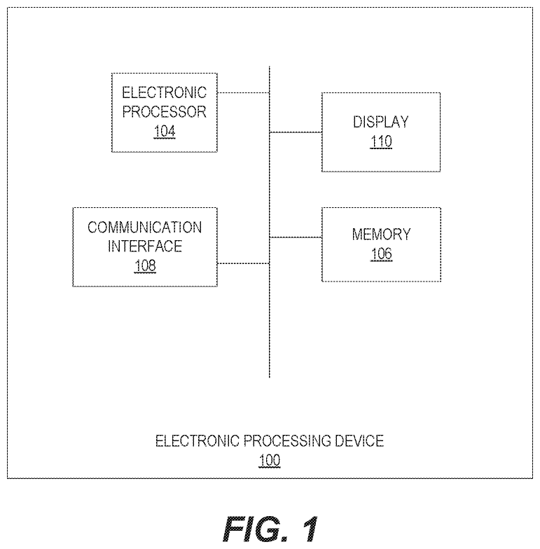

FIG. 1 is a diagram of an example electronic processing device 100. As described more particularly below, the electronic processing device 100 transmits and receives patient data to and from other communication devices and systems (not shown) using either or both one or more of a wired and wireless connection. The electronic processing device 100 communicates directly with other devices, indirectly via a communications network (not shown) or using combinations of both. In some embodiments, the electronic processing device 100 is a handheld device, a stand-alone machine, or a device integrated into a support structure (for example, a medical bed).

In the embodiment illustrated, the electronic processing device 100 includes an electronic processor 104, a non-transitory computer-readable memory 106, an input/output (I/O) or communication interface 108, and a display 110. The illustrated components, along with other various modules and components are coupled to each other by or through one or more electrical connections (for example, control or data buses) that enable communication therebetween. The use of such connections, including control and data buses, for the interconnection between and exchange of information among the various modules and components would be apparent to a person skilled in the art. In some embodiments, the electronic processing device 100 includes fewer or additional components in configurations different from that illustrated in FIG. 1. For example, the electronic processing device 100 may further include an audio speaker, a user interface (for example, a touch-screen through the display 110 or one or more softkeys/buttons), a microphone, and the like.

The electronic processor 104 obtains and provides information (for example, from the memory 106 and/or the communication interface 108), and processes the information by executing one or more software instructions or modules, capable of being stored, for example, in a random access memory (“RAM”) area of the memory 106 or a read only memory (“ROM”) of the memory 106 or another non-transitory computer readable medium (not shown). The software can include firmware, one or more applications, program data, filters, rules, one or more program modules, and other executable instructions. The electronic processor 104 is configured to retrieve from the memory 106 and execute, among other things, software related to the control processes and methods described herein. The memory 106 can include one or more non-transitory computer-readable media, and includes a program storage area and a data storage area. The program storage area and the data storage area can include combinations of different types of memory, as described herein. In the embodiment illustrated, the memory 106 stores, among other things, biometric and clinical data (as described in more detail below), for processing according to the methods described herein.

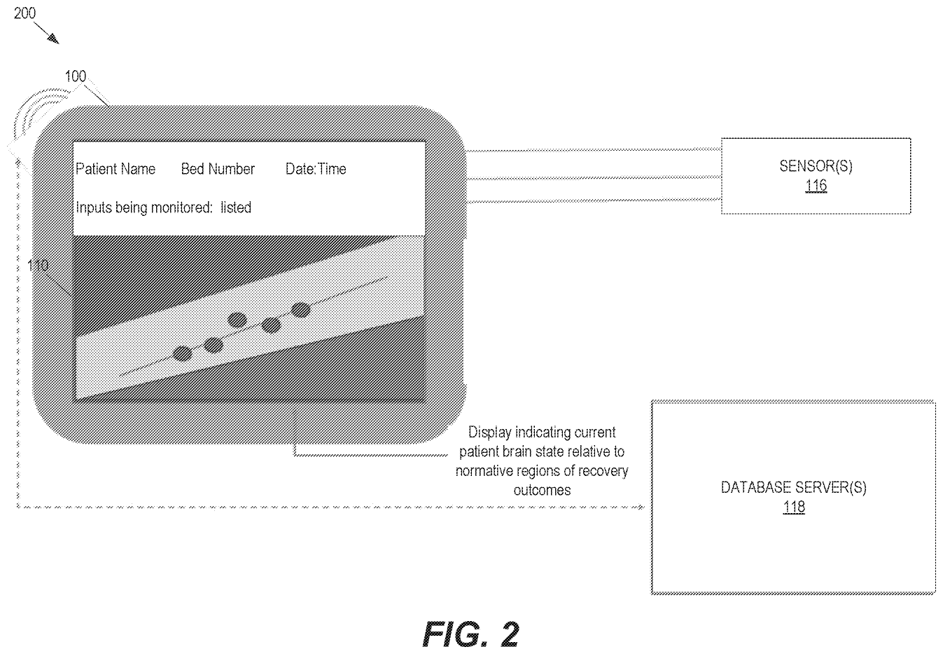

The communication interface 108 receives input from, for example, one or more electronic devices, for example, the biometric sensors 116 and one or more databases 118 (FIG. 2) in communication with the electronic processing device 100, provides system output, or a combination of both. The communication interface 108 obtains information and signals from, and provides information and signals to, (for example, over one or more wired and/or wireless connections) devices both internal and external to the electronic processing device 100. The communication interface 108 may include a transceiver (not shown). Some embodiments include separate transmitting and receiving components, for example, a transmitter and a receiver, instead of a combined transceiver. Output may be provided via the display 110.

The display 110 may include, for example, a liquid crystal display (LCD) touch screen, or an organic light-emitting diode (OLED) touch screen. Alternative embodiments may include other output mechanisms such as, for example, haptic feedback motors and light sources (not shown). Input may be provided via a user interface (not shown), for example, a keypad, a microphone, soft keys, icons, or soft buttons implemented in hardware or presented on the display 110, a scroll ball, buttons, and the like. The communication interface 108 may include a graphical user interface (GUI) (for example, generated by the electronic processor 104, from instructions and data stored in the memory 106, and presented on the display 110) that enables a user to interact with the electronic processing device 100. In some embodiments, additional information to be utilized in the methods described herein may be entered manually via the user interface and/or graphical user interface. As shown in FIG. 2 (described in more detail below) the display 110 is configured to display information relative to the patient including a severity of the injury of the patient and recovery trajectory thereof.

Returning to FIG. 1, the electronic processor 104 is further configured to transmit and receive data to and from the electronic processing device 100. The electronic processor 104 encodes and decodes digital data sent and received by the communication interface 108. The communication interface 108 transmits and receives data to and from, for example, the one or more databases 118. The electronic processor 104 and the communication interface 108 may include various digital and analog components (for example, a transceiver), which for brevity are not described herein and which may be implemented in hardware, software, or a combination of both.

FIG. 2 illustrates a system diagram including the electronic processing device 100 according to some embodiments. The system 200 includes the electronic processing device 100 in communication with one or more biometric sensors 116 and one or more databases 118. The one or more biometric sensors 116 include one or more sensors configured to measure (continuously or discreetly over time) one or more physical characteristics, activity, and/or health status of a patient. The biometric sensor(s) 116 may provide raw biometric data that includes EEG data, intracranial pressure (ICP) data, and electrocardiogram (ECG) data, and the like. The sensor(s) 116 may also be configured to provide indications of health/vitals such as one or more of a captured heart rate, a captured breathing rate, and a captured body temperature of the patient, perhaps accompanying other information. The sensor(s) 116 may include one or more movement sensors (such as an accelerometer, magnetometer, and/or gyroscope) that may periodically or intermittently provide to the electronic processing device 100 indications of orientation, direction, steps, acceleration, and/or speed. The one or more sensor(s) may be standalone biometric devices (for example, a cardiac monitor). In further embodiments, the electronic processing device 100 may include one or more of the sensor(s) 116.

In some embodiments, the sensor(s) 116 may be a wireless device that include its own long-range transceiver and communicates with other communication devices (for example, through one of the databases 118) and/or the electronic processing device 100 directly over a wireless communication channel. The one or more sensors 116 may alternatively be communicatively coupled to the electronic processing device 100 via a physical/wired connection. As explained in more detail below, the one or more biometric sensors 116 provide raw (unprocessed) data to the electronic processor 104 for processing using the methods described herein. The content of the raw biometric data is processed and broken down into several analytic measures/features. The resulting set of analytic measures are categorized by feature and may be stored in a reference data table. Based on an analysis of the particular features and any characteristic shifts of the one or more particular features, a severity of the injury (and recovery trajectory thereof) may be determined.

The one or more network databases 118 may be provided by/housed on a suitable database server communicatively coupled to and accessible by the device 100. The one or more databases 118 may be part of a cloud-based database system. The one or more databases may be accessible directly by the device 100 or over one or more additional networks. In some embodiments, all or part of the database(s) 118 may be locally stored on a local server/network that the electronic processing device 100 may be part of. In some embodiments, as described below, the database(s) 118 electronically store clinical information/data related to the patient. For example, the clinical information may include patient demographics, clinical exam information, pharmaceutical information (past and current medications administered to the patient), type of injury, recent lab information, neuropathology reports, other medical record information, and the like. The one or more databases 118 may also include clinical data of previous patients. For example, the reference data tables described below include EEG data of previous patients.

As described below, the electronic processing device 100 is configured to utilize continuous EEG recordings to identify changes in the EEG signals that confer clinically significant improvement or decline in neuronal function. These changes are compared to previously recorded EEG data of previous patients with similar or the same clinical state (e.g., the same injury) as the current patient to predict the current patient's recovery trajectory. EEG recordings are taken and compared to the reference EEG data continuously so that the recovery trajectory may be recalculated in the case of an unexpected event or change in a patient's vitals or wellbeing.

The device 100 further utilizes data from multiple modalities (for example, text data from patient records, imaging data from imaging studies like MM and Transcranial Doppler (TCD), and biosignal data like EEG and ECG) and machine-learning techniques to evaluate the condition and risk of a brain injury in the patient and calculate the recovery trajectory.

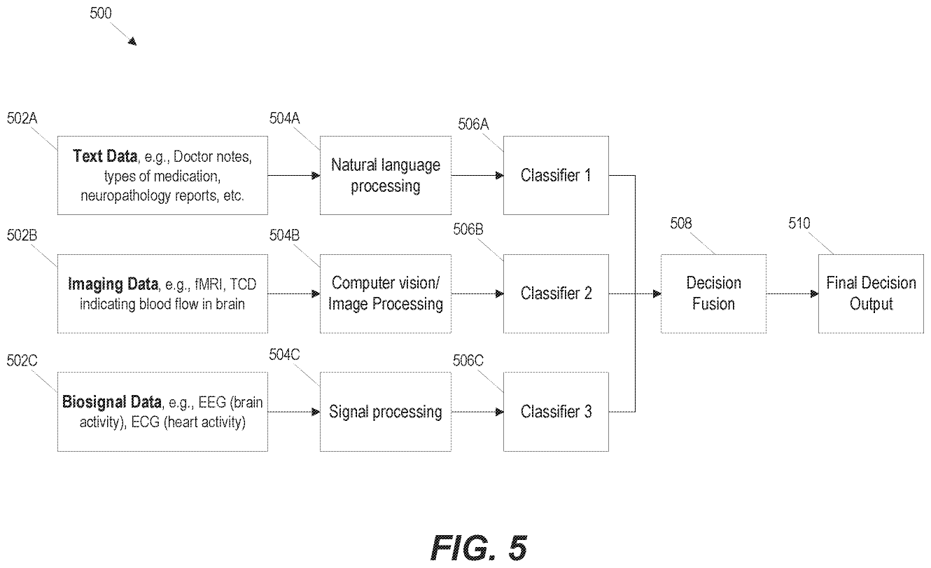

As described in more detail below in regard to FIGS. 5 through 7, the device 100 utilizes machine-learning to analyze data separately for each modality and uses a decision fusion approach to combine the decisions from each modality to evaluate the condition of a brain injury patient. Examples of such analyses are described below:

TEXT DATA ANALYSIS: The disclosed method may utilize text information from sources like patient progress notes, list of medications administered, neuropathology reports etc. to reveal the state of recovery of a brain injury patient. For example, sentiment analysis can be used to automatically scan patient progress report to determine whether the physician expressed a positive, negative or neutral opinion on the progress of the patient.

IMAGING DATA ANALYSIS: Cerebral blood flow in brain injury patients may be visualized using imaging modalities such as fMRI and transcranial Doppler (TCD). Computer vision and image processing techniques can be used to automatically segment the images of cerebral blood flow and compare the segments with images of normal cerebral blood flow.

BIOSIGNAL DATA ANALYSIS: Out of the three modalities, biosignal data may be recorded with the highest temporal resolution, which allows for continuous monitoring of the state of patient. Frequency-based and time-based features are extracted from biosignals and predictive analytics are used to predict the recovery of a brain injury patient. Changes in some frequency-based features at various time points after ischemic stroke may be used to track changes in brain injury state.

As illustrated in FIG. 2, the electronic processing device 100 is coupled to both the one or more sensors 116 and the one or more databases 118 via either or both a wired or wireless connection of the communication interface 108. The wired connection may be implemented, for example via a universal serial bus (USB) connection, a network port and cable connection (for example a category 5 or six, or Cat 5 or Cat 6, connection), and combinations, derivatives, or similar types of connections thereof. The wireless connection may be for a wide area network, for example, the Internet, a local area network, for example, a Bluetooth™ network or Wi-Fi, a Long Term Evolution (LTE) network, a device-to-device network, and combinations or derivatives thereof.

As also illustrated in FIG. 2, the display 110 of the device 100 is configured to display current clinical parameters/data including, for example, patient identifiers (name, bed number, etc.), clinical condition information, last exam information, and biosignals/vitals being monitored. The display 110 also displays a graphic window, within which a graphic model for recovery from the given injury (i.e. a recovery trajectory) will be calculated. In the illustrated embodiment, the trajectory is portrayed on a graph divided into three regions indicative of a condition of the recovery: poor, average, and excellent. The current position of the recovery trajectory of the patient within these regions is continuously updated over time as new clinical and biometric data is retrieved/received. In some embodiments, a best fit line of the recovery trajectory may be calculated and overlaid on the display 110 over the calculated recovery trajectory.

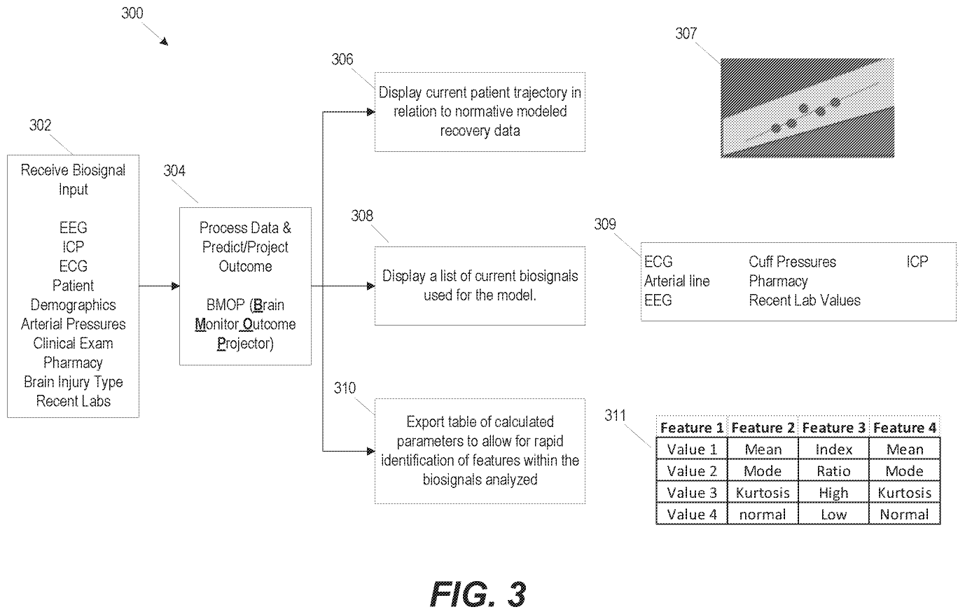

FIG. 3 is a flowchart 300 illustrating an overview of the method of determining a recovery trajectory of a patient based on the severity of an injury implemented by the electronic processing device 100 in accordance to some embodiments. As an example, the methods described herein and illustrated in flowchart 300 are described as being performed by the electronic processing device 100 and, in particular, the electronic processor 104. However, it should be understood that in some embodiments, portions of the methods may be performed by other devices. As previously explained above, the device 100 utilizes a variety of input data (e.g., in step 302) from a variety of sources, including the sensor(s) 116 and the database(s) 118. As also explained above, the information received includes clinical data and biometric sensor data.

The input data is received by the electronic processor 104 (step 302) and processed (step 304). Specifically, the input data is used by the electronic processor 104 to calculate and produce a recovery trajectory indicative of a severity of the injury, for example, using a technique referred to herein as “Brain Monitor Outcome Projector” (BMOP). The electronic processor 104 is configured to determine the recovery trajectory based on one or more stored datasets comparative to the features and their patterns in the particular nature of the injury of the given patient, including the outlook condition (poor, average, and excellent) of the recovery trajectory. As explained in more detail below, the electronic processor 104, during the calculation of the recovery trajectory, processes and breaks down the content of the raw biometric data into several analytic measures/features. The resulting set of analytic measures are categorized by feature and may be stored in a reference data table. Based on an analysis of the particular features and any characteristic shifts of the one or more particular features, a severity of the injury (and recovery trajectory thereof) may be determined.

Following the calculation of the recovery trajectory, the electronic processor 104 displays the determined recovery trajectory on the display 110 (step 306), as previously described above. A graphical example of a displayed recovery trajectory is shown as block 307 in FIG. 3. In some embodiments, the electronic processor 104 may also display, on the display 110, a list of the biometric signals/measurements used in the calculation of the recovery trajectory (step 308). An example of biometric signals/measurements that may be shown on the display 110 in list form is illustrated as block 309 in FIG. 3. In some embodiments, the electronic processor 104 is configured to export the reference data table of analytic measures resulting from the processed raw biometric data to be analyzed for identification of particular known features (step 310). An example of a reference data table is shown as block 311 in FIG. 3.

For example, data streams of vitals may be used for predictions in survival from sepsis, hemorrhage and traumatic brain injury. EEG data has also been found to be prognostic and has been used for monitoring of brain function and injury. Similarly, ICP, as measure by extraventricular drain (EVD) or epidural bolt devices, has also been used to find the optimal perfusion pressure for given brain injured patient. Although not commonly utilized, medical record data such as pharmacy information, imaging information and information about brain injury and current exam also provide relative information useful in calculation of a recovery trajectory.

As previously mentioned above, to allow identification of content without review of raw data, the electronic processor 104 generates a large set of analytic measures derived from the raw biometric data to use in determining the recovery trajectory. The raw biometric data may be broken down/analyzed by various analytic measures from simple characterization of frequency and amplitude to highly complex measures of power, entropy, coherence, symmetry, independent component analysis, rhythmicity and statistical comparisons of variances over time and the like. After the raw biometric data is broken down into a set of analytic measures, the set may be stored in a reference data table. These analytic measures may be compared to similar analytic measures stored within a reference EEG database/data table that includes particular reference waveform data for particular features in order to determine the severity of the patient's injury and recovery trajectory. The electronic processor 104 further utilizes the generated reference data table by storing it and/or modifying the reference EEG database/data table to include the generated data table for use in future queries and calculations of recovery trajectories. As such, the greater the number of analytic measures in any instance/case, the more accurate subsequent queries and cohort development will be, as the stored (reference) analytics will provide greater accuracy in differentiation and alignment between raw data files.

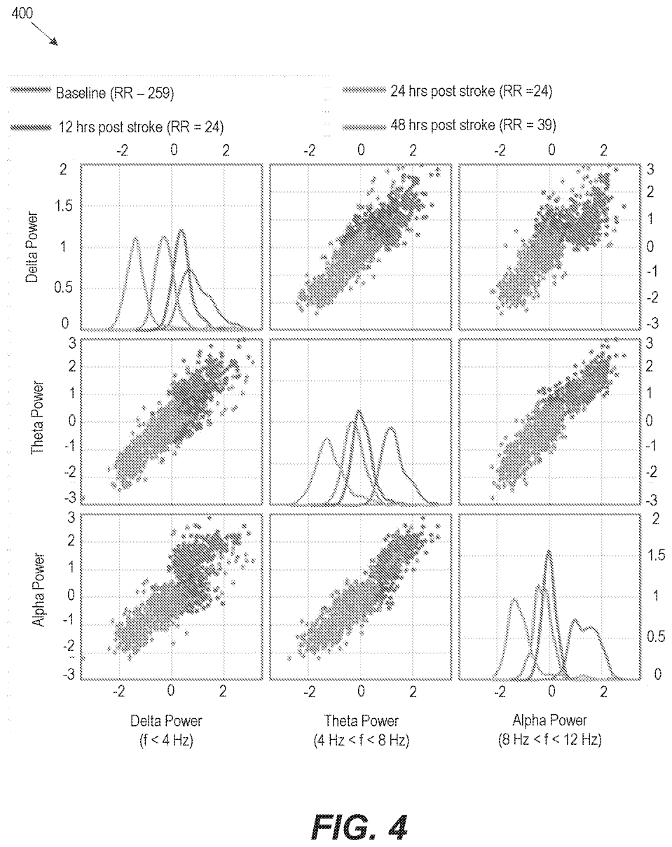

FIG. 4 illustrates a power analysis table 400 illustrating three frequency-based power features extracted from mouse EEG following varied periods of ischemia. Such feature extraction may be used in a machine-learning algorithm of the device 100 during the analysis (e.g., step 304 in FIG. 3). The data illustrated in FIG. 4 suggests that tracking changes in brain injury level over time may be possible through feature extraction. As illustrated in the table 400, three frequency-based power features were extracted from mouse EEG following varied periods of ischemia. These features are derived from the uninjured brain hemisphere of one mouse. These features clearly discriminate between 12, 24, and 48 hours post-stroke. This appears to result from an increase in the power of the faster frequencies over time as would be expected from the clinical recovery course. As illustrated in table 400, this particular analytic provides definite discrimination between different time points post-injury across a spectrum of injury severity. Similar frequency-based analytics are capable of discriminating ischemic changes in human EEG as well.

In some embodiments, content validation may be implemented using the conventional approach to content analysis. For example, in the case of EEG, human interpretation of content may be stored along with the analytic parameters/measures to provide context to the parameters/measures. Once established, the electronic processor 104 can be queried during calculation of the recovery trajectory for files containing specific combinations of analytics that are known to represent specific patterns or content of interest. Validation of successful content identification can then provide additional fields tagging these files as positively containing the target content and accelerating future queries as well as enabling automatic characterization of subsequent data sets added to the database. Over time, the developed reference dataset allows for the production of highly accurate, matched injury datasets for model-building and projections of recovery.

Unlike methods that focus on pattern recognition within the raw data streams, the present methods and processes disclosed herein leverage characteristic changes in a set of analytic measures that are associated with specific underlying changes in the content of the raw data stream. For example, in the case of EEG, a seizure could represent content of interest and may have 50-60 features that develop a reproducible shift in relation to one another that can be used as a marker within the reference dataset for seizure content.

Initially, reference datasets that contain only the target pattern may be analyzed to inform on the pattern of measures associated with the target content. As the reference dataset grows, full-length reference datasets containing content in addition to the target pattern may be analyzed and screened for similar alignment of parameters/features. The electronic processor 104 may utilize one or more processes of pattern confirmation and statistical comparisons of parameter/feature content in the analysis of the surrogate patterns in the analytic measures to determine the recovery trajectory.

It is important to note that in analyzing EEG, often a single feature, for example frequency, is used to assess brain function. However, brain function may be more accurately characterized by combining information from multiple features. As such, the present device 100 may utilize machine-learning, providing an automated, principled approach to combine information from multiple features in order to optimize performance.

FIG. 5 illustrates a diagram 500 of one example of a machine-learning approach implemented by the electronic processing device 100 in accordance with some embodiments. Feature extraction with EEG signals may include manually designed features like alpha-to-delta ratio, peak frequencies, symmetry measures, temporal latency corresponding to a stimulus event, power in frequency bands, auto-regressive coefficients, wavelet coefficients, and the like. In the context of a machine-learning problem, features provide useful information to a classifier and are derived from the raw input data. These features are used by the classifier to label the data, i.e. to indicate changes in brain injury level. Since brain injury patients are monitored continuously using the systems and methods described herein, the outputs of the algorithms are updated as new data becomes available. The rate of data acquisition may differ for each data type as different data streams from multiple modalities are utilized. For example, text data like patient progress reports may be updated on a daily basis, imaging studies may be conducted once every couple of days, and biosignal data may be updated every few seconds.

In the example of FIG. 5, data is provided in three data streams: text data 502A (e.g., doctors notes, types of medications, neuropathology reports, etc.), imaging data (e.g., MRI, CT, TCD, etc.), and biosignal data 502C (e.g., EEG and ECG signals). Relevant features are extracted from the data streams using, for example, natural language processing (step 504A), computer vision/image processing (step 504B), and signal processing (step 504C), respectively, and are used to train the respective classifier 506A, 506B, 506C for each data stream. The classifiers 506A, 506B, 506C are each applied to the respective data stream to predict the extent of recovery from brain injury in the patient. For example, each classifier 506A, 506B, 506C may be trained to predict a “class” (i.e., Class 1 through Class 5), where the value of the class is indicative of the current severity of the injury in the patient. The output of the classifiers 506A, 506B, 506C are then fused/combined via a classifier/decision fusion 508 in order to produce a final decision/diagnosis 510 regarding the predicted recovery of the patient.

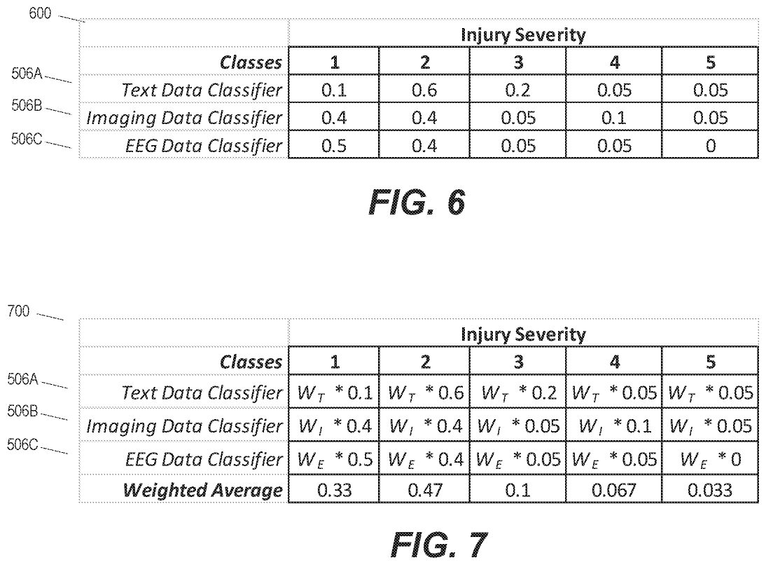

In some implementations, state-of-the-art classifiers (for example, support vector machines (SVM), neural networks and random forests) are used to predict the recovery trajectory of the patient of the brain injury. In some implementations, each of the classifiers 506A, 506B, 506C is configured to output a relative probability for each “class” (i.e., Class 1 through Class 5) that the current severity of the injury falls into each respective class. FIG. 6 is a table 600 illustrating an example of the probability prediction output by each classifier 506A, 506B, 506C based on the respective data streams. provide a measure of probability of predicting each class at the output for a given data sample. Note, that since each classifier 506A, 506B, 506C outputs a probability measure, the values in each row sum to one.

In some implementations, the probability outputs from the classifiers 506A, 506B, 506C are combined using a weighted majority voting scheme. In the weighted majority voting scheme, the probability outputs of each classifier 506A, 506B, 506C is multiplied by a weight parameter and then the average of the weighted probabilities for each “class” is calculated. The class with the highest weighted average probability is chosen as the final decision (i.e., the final decision output 510 in FIG. 5). FIG. 7 is a table 700 illustrating one example of a weighted majority voting scheme using the probability values of FIG. 6. Each of the classifiers 506A, 506B, 506C are assigned a weight W_{T}, W_{I}, and W_{E} respectively. The probability value determined by each classifier for each severity class is multiplied by the weight value for the respective classifier/data stream and an average of the weighted probability values from all of the classifiers for each individual severity class is calculated. In some implementations, the severity class with the highest weighted average probability value is selected and output by the system as the final decision output of the current severity of the injury.

In the example of FIG. 7, the weights W_{T}, W_{I}, and W_{E} are each set equal to one. In some implementations, the values of the weights may be adjustable and may be changed manually (for example, via one or more user inputs of the communication interface 108 of the electronic processing device 100). In other implementations, the values of the weights may be adjusted automatically (for example, based on prior feedback regarding the relative accuracy of each classifier for a particular patient (or category of patients) or based on a current state or activity level of the patient). For example, when the patient is moving, the EEG data may be corrupted by motion artifacts, thereby reducing the reliability of the EEG data classifier. In this case, a greater weight may be assigned to the text data classifier weight W_{T}, and a lower weight to the EEG data classifier W_{E}, as movement of the patient may have little or no impact on the reliability of the text data and the output of the text data classifier.

The machine-learning approach is configured to update its parameters used in determining the recovery trajectory based on the new acquired data to provide a reliable evaluation of patient condition. The machine-learning approach is also configured to fuse information across the different time scales in order to provide predictions of the current brain state.

In the foregoing specification, specific embodiments have been described. However, one of ordinary skill in the art appreciates that various modifications and changes may be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a,” “has . . . a,” “includes . . . a,” or “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more generic or specialized electronic processors (or “processing devices”) such as microprocessors, digital signal processors, customized processors and field programmable gate arrays (FPGAs) and unique stored program instructions (including both software and firmware) that control the one or more electronic processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

What is claimed is:

1. A device configured to calculate a recovery trajectory of a patient based on a severity of an injury of the patient, the device comprising:
a communication interface;
a display;
an electronic processor configured to
receive, via the communication interface, clinical information regarding the patient,
receive, via the biometric sensor, raw biometric data,
analyze the raw biometric data and the clinical information to produce a set of analytic measures,
calculate, based on the clinical information and the set of analytic measures, a recovery trajectory, and
display, on the display, the recovery trajectory.

2. The device of claim 1, wherein the electronic processor is configured to produce the set of analytic measures based on a predetermined content within the raw biometric data related to the injury and a characteristic shift of one or more features within the raw biometric data.

3. The device of claim 1, wherein the electronic processor is configured to analyze, via machine learning, the set of analytic measures and clinical information to calculate the recovery trajectory.

4. The device of claim 1, wherein the raw biometric data includes at least one selected from the group consisting of electroencephalographic data, intracranial pressure data, and electrocardiogram data.

5. The device of claim 1, wherein the raw biometric data is a real-time continuous feed.

6. The device of claim 1 wherein the electronic processor is further configured to recalculate the recovery trajectory in response to receiving either or both new clinical information and new raw biometric data.

7. The device of claim 1, wherein the electronic processor is configured to calculate the recovery trajectory by
comparing the analytic measures and clinical information for the patient to historical data for other patients;
identifying a trend in changes to a determined severity of the injury of the patient over a period of time; and
determining a predicted recovery outcome based on the comparison and the identified trend.

8. The device of claim 7, wherein the electronic processor is configured to display the recovery trajectory by displaying a graph on the display divided into three regions including a first region indicative of poor recovery conditions, a second region indicative of average recovery conditions, and a third region indicative of excellent recovery,
wherein the graph is indicative of monitored changes in the severity of the injury and predicted future severity conditions relative to the three regions.

9. The device of claim 1, wherein the injury of the patient includes a brain injury, and wherein the electronic processor is configured to receive raw biometric data by receiving a continuous stream of EEG data for the patient.

10. The device of claim 1, wherein the electronic controller is configured to calculate the recovery trajectory by
receiving a plurality of data streams for the patient including a text data stream providing text-format clinical information for the patient and a biosignal data stream providing raw biometric data for the patient from the biometric sensor,
applying a separate machine-learning classifier of a plurality of machine-learning classifiers to each data stream of the plurality of data streams, wherein each machine-learning classifier is configured to produce an output indicating a relative probability for each of a plurality of severity classes for the injury, and
determining a recovery trajectory for the patient based at least in part on an averaging of the probability for each of the severity classification from each of the plurality of machine-learning classifiers.

11. A method for calculating a recovery trajectory of a patient based on a severity of an injury of the patient, the method comprising:
receiving, via a communication interface, clinical information regarding the patient;
receiving, via a biometric sensor, raw biometric data;
analyzing the raw biometric data and the clinical information to produce a set of analytic measures;
calculating, based on the clinical information and the set of analytic measures, a recovery trajectory; and
displaying, on a display, the recovery trajectory.

12. The method of claim 11, wherein the set of analytic measures is produced based on a predetermined content within the raw biometric data related to the injury and a characteristic shift of one or more features within the raw biometric data.

13. The method of claim 11, further comprising analyzing, via machine learning, the set of analytic measures and clinical information to calculate the recovery trajectory.

14. The method of claim 11, wherein the raw biometric data includes at least one selected from the group consisting of electroencephalographic data, intracranial pressure data, and electrocardiogram data.

15. The method of claim 11, wherein the raw biometric data is a real-time continuous feed.

16. The method of claim 11, further comprising recalculating the recovery trajectory in response to receiving either or both new clinical information and new raw biometric data.

17. The method of claim 11, wherein calculating the recovery trajectory includes
comparing the analytic measures and clinical information for the patient to historical data for other patients;
identifying a trend in changes to a determined severity of the injury of the patient over a period of time; and
determining a predicted recovery outcome based on the comparison and the identified trend.

18. The method of claim 17, wherein displaying the recovery trajectory includes displaying a graph on the display divided into three regions including a first region indicative of poor recovery conditions, a second region indicative of average recovery conditions, and a third region indicative of excellent recovery,
wherein the graph is indicative of monitored changes in the severity of the injury and predicted future severity conditions relative to the three regions.

19. The method of claim 11, wherein the injury of the patient includes a brain injury, and wherein receiving the raw biometric data includes receiving a continuous stream of EEG data for the patient.

20. The method of claim 11, wherein calculating the recovery trajectory includes
receiving a plurality of data streams for the patient including a text data stream providing text-format clinical information for the patient and a biosignal data stream providing raw biometric data for the patient from the biometric sensor,
applying a separate machine-learning classifier of a plurality of machine-learning classifiers to each data stream of the plurality of data streams, wherein each machine-learning classifier is configured to produce an output indicating a relative probability for each of a plurality of severity classes for the injury, and
determining a recovery trajectory for the patient based at least in part on an averaging of the probability for each of the severity classification from each of the plurality of machine-learning classifiers.
